(19) United States
(12) Patent Application Publication
(10) Pub. No.: US 2023/0414495 A1
(43) Pub. Date: Dec. 28, 2023

(54) PUNCTAL PLUGS CONTAINING MICELLE-ENCAPSULATED OPHTHALMIC PHARMACEUTICALS AND METHODS FOR MAKING THEREOF

(71) Applicant: EXIMORE LTD., Jerusalem (IL)

(72) Inventors: Eyal SHEETRIT, Jerusalem (IL); Guy TOMER, Modiin (IL)

(21) Appl. No.: 18/035,816

(22) PCT Filed: Nov. 8, 2021
(86) PCT No.: PCT/IB2021/000790

§ 371 Date: May 8, 2023

Related U.S. Application Data
(60) Provisional application No. 63/111,199, filed on Nov. 9, 2020.

(51) Int. Cl. A61K 9/00; A61K 9/107; A61K 47/02; A61K 47/34; A61K 45/06; A61K 9/19

(52) U.S. Cl. CPC A61K 9/0051; A61K 9/1075; A61K 47/02; A61K 47/34; A61K 45/06; A61K 9/19

(57) ABSTRACT

Embodiments of the disclosure provide devices containing micelle-encapsulated ophthalmic pharmaceuticals. Some embodiments are directed to punctal plug devices comprising: a plurality of micelles containing an active pharmaceutical ingredient; a quantity of fumed silica; and a quantity of a binder. In some embodiments, the disclosure provides methods of producing or making the punctal plugs described here.

### CROSS REFERENCE TO RELATED APPLICATION

This application is a continuation of and claims priority from co-ending U.S. Provisional Application No. 63/111,199, filed on Nov. 9, 2020, entitled “PUNCTAL PLUGS CONTAINING MICELLE-ENCAPSULATED OPHTHALMIC PHARMACEUTICALS AND METHODS FOR MAKING THEREOF,” which is herein incorporated by reference in its entirety.

### FIELD OF THE INVENTION

The exemplary embodiments relate to devices for the administration of active pharmaceutical ingredients (“APIs”). More particularly, the exemplary embodiments relate to punctal plugs containing ophthalmic APIs encapsulated in micelles, and methods for using such devices.

### BACKGROUND

The human eye is a complex organ with intricate anatomical and physiological barriers. The anterior segment of the eye consists of the cornea, conjunctiva, aqueous humor, iris, ciliary body, and lens. Common diseases affecting the anterior segment of the eye are dry eye syndrome, glaucoma, allergic conjunctivitis, anterior uveitis, and cataract. It is often desirable to administer APIs to the eye for these and other diseases. However, factors such as tear secretions and internal blood circulation present barriers that prevent APIs from reaching the desired location within the eye.

### SUMMARY

In some embodiments, a device includes a quantity of micelles or a plurality of micelles containing an active pharmaceutical ingredient; a quantity of fumed silica; and a quantity of an epoxy, wherein the device is in the form of a punctal plug, and wherein the device includes between 5% and 30% of the active pharmaceutical ingredient by weight.

In some embodiments, the device also includes a quantity of a clay. In some embodiments, the clay is kaolin.

In some embodiments, a method includes providing micelles or a plurality of micelles containing an active pharmaceutical ingredient; suspending fumed silica in an alcohol to produce a suspension; drying the suspension to produce dried silica; milling the dried silica to produce milled silica; mixing the micelles, the milled silica, and an epoxy resin to produce a paste; placing the paste in a mold; and curing the molded paste to produce punctal plugs.

In some embodiments, the step of providing the micelles containing the active pharmaceutical ingredient includes sub-steps of preparing a dispersion of a surfactant in a buffer; dissolving the active pharmaceutical ingredient in an alcohol to produce an active pharmaceutical ingredient solution; mixing the active pharmaceutical ingredient solution with the dispersion and adding an additional quantity of an alcohol to produce a mixture; stirring the mixture until the alcohol evaporates to produce the mixture without alcohol; and freezing the mixture without alcohol and lyophilizing to produce the micelles containing the active pharmaceutical ingredient.

### BRIEF DESCRIPTION OF THE DRAWINGS

FIG. 1 shows release of tacrolimus (Tac) from exemplary plugs with and without micelles illustrated by a graph of a mass of tacrolimus released per day by two exemplary embodiments of punctal plugs: Plug+Tac; and Plug+Tac/micelles.

FIG. 2 shows accumulation of tacrolimus (Tac) released from exemplary plugs with and without micelles illustrated by a graph of the accumulation of percentage of total mass of tacrolimus (Tac) released by two exemplary embodiments of punctal plugs: Plug+Tac; and Plug+Tac/micelles.

FIG. 3 shows accumulation of tacrolimus (Tac) released from exemplary plugs with and without micelles illustrated by a graph of the accumulation of total mass of tacrolimus released by two exemplary embodiments of punctal plugs: Plug+Tac; and Plug+Tac/micelles.

### DETAILED DESCRIPTION

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though they may. Furthermore, the phrases “in another embodiment” and “in some other embodiments” as used herein do not necessarily refer to a different embodiment, although they may. Thus, as described below, various embodiments of the invention may be readily combined, without departing from the scope or spirit of the invention.

The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.” Spatial or directional terms, such as “left”, “right”, “inner”, “outer”, “above”, “below”, and the like, are not to be considered as limiting as the invention can assume various alternative orientations. All numbers used in the specification are to be understood as being modified in all instances by the term “about”. The term “about” means a range of plus or minus ten percent of the stated value.

Unless otherwise indicated, all ranges or ratios disclosed herein are to be understood to encompass any and all subranges or sub-ratios subsumed therein. Unless otherwise indicated, all ranges or ratios herein are understood to be inclusive (i.e., to include both the minimum and maximum values of such ranges or ratios). For example, a stated range or ratio of “1 to 10” should be considered to include any and all subranges between (and inclusive of) the minimum value of 1 and the maximum value of 10; that is, all subranges or sub-ratios beginning with a minimum value of 1 or more and ending with a maximum value of 10 or less, such as but not limited to, 1 to 6.1, 3.5 to 7.8, and 5.5 to 10.

The exemplary embodiments relate to punctal plugs that are suitable for placement within the tear duct of the human eye, and which contain therein ophthalmic APIs encapsulated within micelles. The exemplary embodiments also relate to methods for producing micelles having ophthalmic APIs encapsulated therein, and to methods for producing punctal plugs containing such micelles therein.

In some embodiments, a method for producing micelles containing an ophthalmic API includes preparing a dispersion of a surfactant in a buffer; dissolving an API in an alcohol to produce an API solution; mixing the API solution with the dispersion and adding an additional quantity of an alcohol to produce a mixture; stirring the mixture until the alcohol evaporates to produce the mixture without alcohol; and freezing the mixture without alcohol and lyophilizing to produce micelles containing the ophthalmic API.

In some embodiments, a method for producing a plurality of micelles containing an ophthalmic API and chitosan includes preparing a dispersion of a surfactant in a buffer; preparing a solution of chitosan in an acid to produce a chitosan solution; mixing the dispersion with the chitosan solution to produce a surfactant/chitosan mixture; dissolving an API in an alcohol to produce an API solution; mixing the API solution with the surfactant/chitosan mixture and adding an additional quantity of an alcohol to produce an API/surfactant/chitosan mixture; stirring the API/surfactant/chitosan mixture until the alcohol evaporates to produce the API/surfactant/chitosan mixture without alcohol; and freezing the API/surfactant/chitosan mixture without alcohol and lyophilizing to produce micelles containing the ophthalmic API.

In some embodiments, a method for producing micelles including an ophthalmic API includes dissolving an API and a block copolymer in aqueous medium at or above a critical micelle concentration to produce a solution, and dehydrating the solution. In some embodiments, the aqueous medium is distilled deionized water. In some embodiments, the aqueous medium is a buffer.

In some embodiments, a method for producing micelles including an ophthalmic API includes dissolving an API and an amphiphilic copolymer in a volatile organic solvent to produce a solution, evaporating the volatile organic solvent to produce a thin film of the API and the amphiphilic copolymer, and reconstituting the thin film with water to produce micelles.

In some embodiments, a method for producing micelles including an ophthalmic API includes dissolving an API and a polymer in an organic solvent to produce a solution; placing the solution in a dialysis bag; and immersing the dialysis bag in water, whereby the organic solvent is exchanged with water through the dialysis bag, thereby producing micelles.

In some embodiments, a method for producing punctal plugs including an API includes dissolving an API in an alcohol to produce an API solution; mixing the API solution with fumed silica to produce an API/silica mixture; drying the API/silica mixture to produce a dried API/silica mixture; milling the dried API/silica mixture to produce a milled API/silica mixture; mixing the milled API/silica mixture with an epoxy resin to produce a paste; placing the paste in a mold; and curing the molded paste to produce punctal plugs. In some embodiments, the paste is produced by mixing the milled API/silica mixture, the epoxy resin, and a clay. In some embodiments, the clay is kaolin.

In some embodiments, a method for producing punctal plugs including API-loaded micelles suspending fumed silica in an alcohol; drying the suspension to produce dried silica; milling the dried silica to produce milled silica; mixing API-loaded micelles, the milled silica, and an epoxy resin to produce a paste; placing the paste in a mold; and curing the molded paste to produce punctal plugs.

In some embodiments, the surfactant is a hydrophilic surfactant. In some embodiments, the surfactant is a non-ionic surfactant. In some embodiments, the surfactant is a poloxamer. In some embodiments, the surfactant is poloxamer 407. In some embodiments, the surfactant is the hydrophilic non-ionic surfactant commercialized by BASF SE of Ludwigshafen, Germany under the trade name Pluronic F127.

In some embodiments, the buffer includes an acid. In some embodiments, the buffer includes acetic acid. In some embodiments, the buffer includes acetic acid at pH 4.5.

In some embodiments, the API is tacrolimus. In some embodiments, the API is an immunosuppressant such as cyclosporine or tacrolimus. In some embodiments, the API is a prostaglandin analog suitable for ophthalmic use, such as latanoprost, travoprost, bimatoprost, or tafluprost. In some embodiments, the API is a corticosteroid such as dexamethasone, fluorometholone, triamcinolone, or triamcinolone acetonide. In some embodiments, the API is a non-steroidal anti-inflammatory drug (“NSAID”) such as indomethacin, diclofenac sodium, lornoxicam, or ketorolac. In some embodiments, the API is an anti-glaucoma drug such as pilocarpine, ethoxzolamide, metipranolol, or latrunculin A. In some embodiments, the API is an antioxidant such as tocopherol, curcumin, or a-lipoic acid. In some embodiments, the API is an antifungal such as terbinafine, itraconazole, sirolimus, or sertaconazole. In some embodiments, the API is an antiviral such as cidofovir or acyclovir. In some embodiments, the API is an ophthalmic API that has low water solubility and/or is hydrophobic, such as ciprofloxacin, itraconazole, diclofenac, fluconazole, dexamethasone, cyclosporine A, muscone, cidofovir, a-tocopherol, ketorolac, curcumin, lornoxicam, or carbamazepine. In some embodiments, the API is pirenzepine hydrochloride, genistein, spironolactone, or imatinib. In some embodiments, more than one API is used (e.g., more than one of the exemplary APIs listed above).

In some embodiments, the alcohol is ethanol.

In some embodiments, a punctal plug includes an API adsorbed directly to fumed silica (e.g., not in the form of micelles) as described above and also an API loaded into micelles as described above. In such embodiments, the API loaded into micelles would be released from the punctal plug more rapidly, while the API adsorbed directly to fumed silica would be released from the punctal plug more slowly. Consequently, in such embodiments, the sustained release profile of the API can be tuned by adjusting the ratio between the two types of the API. In some embodiments, an exemplary punctal plug also includes micronized particles of an API. In such embodiments, the micronized particles of the API would release more slowly than either the API adsorbed directly to the fumed silica or the API loaded into micelles due to the crystallinity and size of the micronized particles. Consequently, in such embodiments, the sustained release profile of the API could be further tuned.

In some embodiments, an exemplary punctal plug includes at least one additive. In some embodiments, the at least one additive includes a biocompatible plasticizer included so as to make the punctal plug more flexible and less brittle, to facilitate its removal from the mold intact, and to reduce the risk of injuries to the punctum and lacrimal canal tissues as a result of a tough and non-flexible biomaterial. In some embodiments, the plasticizer includes polyethylene glycol, a glycerol, a polyol, a surfactant, or combinations thereof. In some embodiments, the exemplary punctal plug includes a plasticizer at a quantity of between 1% and 20% by weight. In some embodiments, the plasticizer includes polyethylene glycol 400 at a quantity of between 5% and 15% by weight.

In some embodiments, an exemplary punctal plug prepared as described herein is dry and shelf-stable. In some embodiments, an exemplary punctal plug prepared as described herein includes micelles loaded with an API, fumed silica, and a binder. In some embodiments, the binder is an epoxy. In some embodiments, an exemplary punctal plug prepared as described herein, once re-wet, provides sustained release of an API for at least 60 days.

In some embodiments, an exemplary punctal plug prepared as described herein includes between 20% and 60% of a binder by dry weight (e.g., 21-59; 22-58; 23-57; 24-56; 25-26-54; 27-53; 28-52; 29-51; 30-50; 31-49; 32-48; 33-47; 34-46; 35-45; 36-44; 37-43; 38-42; 39-41); 20% or more of a binder by dry weight (e.g., 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60); or 60% or less of a binder by dry weight (e.g., 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20). In some embodiments, an exemplary punctal plug prepared as described herein includes between 20% and 50% of a binder by dry weight. In some embodiments, an exemplary punctal plug prepared as described herein includes between 20% and 40% of a binder by dry weight. In some embodiments, an exemplary punctal plug prepared as described herein includes between 20% and 30% of a binder by dry weight. In some embodiments, an exemplary punctal plug prepared as described herein includes between 30% and 60% of a binder by dry weight. In some embodiments, an exemplary punctal plug prepared as described herein includes between 30% and 50% of a binder by dry weight. In some embodiments, an exemplary punctal plug prepared as described herein includes between 30% and 40% of a binder by dry weight. In some embodiments, an exemplary punctal plug prepared as described herein includes between 40% and 60% of a binder by dry weight. In some embodiments, an exemplary punctal plug prepared as described herein includes between 40% and 40% of a binder by dry weight. In some embodiments, an exemplary punctal plug prepared as described herein includes between 50% and 60% of a binder by dry weight.

In some embodiments, an exemplary punctal plug prepared as described herein includes between 5% and 15% of a clay by dry weight (e.g., 6-14; 7-13; 8-12; 9-11); 5% or more of a clay by dry weight (e.g., 5.5, 6, 6.5 7, 7.5, 8, 8.5, 9, 9.5, 10, 10.5, 11, 11.5 12, 12.5, 13, 13.5, 14, 14.5, 15, 15.5); or 15% or less of a clay by dry weight (e.g., 14.5, 14, 13.5, 13, 12.5, 12, 11.5, 11, 10.5, 10, 9.5, 9, 8.5, 8, 7.5, 7, 6.5, 6, 5.5, 5, 4.5). In some embodiments, an exemplary punctal plug prepared as described herein includes between 5% and 12.5% of a clay by dry weight. In some embodiments, an exemplary punctal plug prepared as described herein includes between 5% and 10% of a clay by dry weight. In some embodiments, an exemplary punctal plug prepared as described herein includes between 5% and 7.5% of a clay by dry weight. In some embodiments, an exemplary punctal plug prepared as described herein includes between 7.5% and 15% of a clay by dry weight. In some embodiments, an exemplary punctal plug prepared as described herein includes between 7.5% and 12.5% of a clay by dry weight. In some embodiments, an exemplary punctal plug prepared as described herein includes between 7.5% and 10% of a clay by dry weight. In some embodiments, an exemplary punctal plug prepared as described herein includes between 10% and 15% of a clay by dry weight. In some embodiments, an exemplary punctal plug prepared as described herein includes between 10% and 12.5% of a clay by dry weight. In some embodiments, an exemplary punctal plug prepared as described herein includes between 12.5% and 15% of a clay by dry weight.

In some embodiments, an exemplary punctal plug prepared as described herein includes between 10% and 30% of fumed silica by dry weight (e.g., 10-29; 11-28; 12-27; 13-26; 14-25; 15-24; 16-23; 17-22; 18-21; 19-20); 10% or more of fumed silica by weight (e.g., 11, 12, 13, 14, 15, 16, 17, 18, 19, 20, 21, 22, 23, 24, 25, 26, 27, 28, 29, 30); or 30% or less of fumed silica by dry weight (e.g., 29, 28, 27, 26, 25, 24, 23, 22, 21, 20, 19, 18, 17, 16, 15, 14, 13, 12, 11, 10, 9, 8, 7, 6, 5, 4, 3, 2, 1). In some embodiments, an exemplary punctal plug prepared as described herein includes between 10% and 25% of fumed silica by dry weight. In some embodiments, an exemplary punctal plug prepared as described herein includes between 10% and 20% of fumed silica by dry weight. In some embodiments, an exemplary punctal plug prepared as described herein includes between 10% and 15% of fumed silica by dry weight. In some embodiments, an exemplary punctal plug prepared as described herein includes between 15% and 30% of fumed silica by dry weight. In some embodiments, an exemplary punctal plug prepared as described herein includes between 15% and 25% of fumed silica by dry weight. In some embodiments, an exemplary punctal plug prepared as described herein includes between 15% and 20% of fumed silica by dry weight. In some embodiments, an exemplary punctal plug prepared as described herein includes between 20% and 30% of fumed silica by dry weight. In some embodiments, an exemplary punctal plug prepared as described herein includes between 20% and 25% of fumed silica by dry weight. In some embodiments, an exemplary punctal plug prepared as described herein includes between 25% and 30% of fumed silica by dry weight.

In some embodiments, an exemplary punctal plug prepared as described herein includes between 5% and 30% of an API by dry weight (e.g., 6-29; 7-28; 8-27; 9-26; 10-25; 11-24; 12-23; 13-22; 14-21; 15-20; 16-19; 17-18); 5% or more of an API by dry weight (e.g., 6, 7, 8, 9, 10, 11, 12, 13, 14, 15, 16, 17, 18, 19, 20, 21, 22, 23, 24, 25, 26, 27, 28, 29, 30); or 30% or less of an API by dry weight (e.g., 29, 28, 27, 26, 25, 24, 23, 22, 21, 20, 19, 18, 17, 16, 15, 14, 13, 12, 11, 10, 9, 8, 7, 6, 5, 4, 3, 2, 1). In some embodiments, an exemplary punctal plug prepared as described herein includes between 5% and 25% of an API by dry weight. In some embodiments, an exemplary punctal plug prepared as described herein includes between 5% and 20% of an API by dry weight. In some embodiments, an exemplary punctal plug prepared as described herein includes between 5% and 15% of an API by dry weight. In some embodiments, an exemplary punctal plug prepared as described herein includes between 5% and 10% of an API by dry weight. In some embodiments, an exemplary punctal plug prepared as described herein includes between 10% and 30% of an API by dry weight. In some embodiments, an exemplary punctal plug prepared as described herein includes between 10% and 25% of an API by dry weight. In some embodiments, an exemplary punctal plug prepared as described herein includes between 10% and 20% of an API by dry weight. In some embodiments, an exemplary punctal plug prepared as described herein includes between 10% and 15% of an API by dry weight. In some embodiments, an exemplary punctal plug prepared as described herein includes between 15% and 30% of an API by dry weight. In some embodiments, an exemplary punctal plug prepared as described herein includes between 15% and 25% of an API by dry weight. In some embodiments, an exemplary punctal plug prepared as described herein includes between 15% and 20% of an API by dry weight. In some embodiments, an exemplary punctal plug prepared as described herein includes between 20% and 30% of an API by dry weight. In some embodiments, an exemplary punctal plug prepared as described herein includes between 20% and 25% of an API by dry weight. In some embodiments, an exemplary punctal plug prepared as described herein includes between 25% and 30% of an API by dry weight.

### Example 1: Preparation of Pluronic Micelles Containing Tacrolimus

A dispersion of Pluronic F-127 was prepared by dissolving 1.5 g of F-127 in 15 mL acetic acid buffer (pH 4.5) for 48 h at 48° C., then adding another 15 mL of acetic acid buffer (pH 4.5).

0.642 g of tacrolimus was dissolved in a minimal volume of ethanol, about 1.5 mL (1:2 w/v, respectively), then added to the F-127 dispersion, followed by an additional 50 mL ethanol. The mixture was stirred overnight at room temperature in the chemical hood, until complete evaporation of ethanol.

The resulting micelles were frozen in liquid nitrogen and lyophilized overnight.

### Example 2: Preparation of Pluronic Micelles Containing Tacrolimus and Chitosan

A dispersion of Pluronic F-127 was prepared by dissolving 1.5 g of F-127 in 15 mL acetic acid buffer (pH 4.5) for 48 hours at 48° C., then adding another 15 mL of acetic acid buffer (pH 4.5).

Chitosan (“CH”) solution (1%, w/v) was prepared by adding 30 mg chitosan to 3 mL aqueous acetic acid (0.5%, v/v), and stirring for 24 hours at room temperature.

An F-127/CH mixture (5% & 0.1% w/v, respectively) was prepared by adding the chitosan solution (3 mL of 1%, w/v) to the F-127 dispersion (27 mL of 5.56% w/v).

0.642 g of tacrolimus was dissolved in a minimal volume of ethanol, about 1.5 mL (1:2 w/v, respectively), then added to the F-127/CH mixture, followed by 50 mL ethanol. The mixture was stirred overnight at room temperature in the chemical hood, until complete evaporation of ethanol.

The resulting micelles were frozen in liquid nitrogen and lyophilized overnight.

### Example 3: Analysis of the Drug Content of the Micelles

In order to extract tacrolimus from the micelles, 20 μL of ethanol was added to 10 mg of lyophilized micelles, followed by 100 μL of acetonitrile.

The mixture was centrifuged for 10 minutes at 12,000 rpm. The supernatant was transferred to a glass insert and injected to a high-performance liquid chromatography (“HPLC”) machine. The HPLC was configured as follows:
Column: C18 column (Phenomenex, 4.6 mm×250 mm, 5 um)
Mobile phase: Acetonitrile/0.1% phosphoric acid, 90:10 v:v
10 μL injection volume, 1 mL/min flow, 10 min run time
Tacrolimus peak retention time ˜6.5 min
Detection wavelength 220 nm
Drug loading (“DL”) and entrapment efficiency (“EE”) were calculated as follows:

$$DL ( \% ) = \frac{Amount of drug}{Amount of polymer + drug} \times 100$$

$$EE ( \% ) = \frac{Measured drug loading}{Theoretical drug loading} \times 100$$

By the foregoing method, micelles containing tacrolimus produced in accordance with Example 1 above were found to have drug loading of 18.6% and encapsulation efficiency of 22%. Also by the foregoing method, micelles containing tacrolimus and chitosan produced in accordance with Example 2 above were found to have drug loading of 25.5% and encapsulation efficiency of 24%.

### Example 4: Preparation of Composite Material and Punctal Plugs Containing Free Tacrolimus

0.45 grams of tacrolimus was dissolved in 28.5 grams of ethanol, and the solution was added to 0.3 grams of fumed silica. The mixture was allowed to dry in the fumed hood overnight. The dried tacrolimus/silica mixture was milled using a pestle and mortar. In some embodiments, the mixture was milled to an average particle size of about 150 microns. 0.281 grams of the dried and milled tacrolimus/silica mixture were mixed with 0.112 grams of kaolin and 0.1675 grams of an epoxy resin (Epo-TEK 301) and the mixture was mixed thoroughly until a homogenous paste was achieved.

The paste was put into a mold and left to cure at room temperature under pressure applied by a hydraulic bench press at 6-8 tons of pressure for 2 days. The resulting plugs were released from the mold.

Tacrolimus constituted 30% of the finished plug by dry weight.

### Example 5: Preparation of Composite Material and Punctal Plugs Containing Tacrolimus-Loaded Micelles

0.2 grams of fumed silica was resuspended in 19 grams of ethanol, and dried overnight in the fumed hood. The dried silica was milled using a pestle and mortar. 0.25 grams of lyophilized tacrolimus-loaded micelles, which were prepared in accordance with Example 1 above, and 0.1475 grams of epoxy resin (Epo-TEK 301) were added to 0.1 gram of dried milled fumed silica, and the mixture was mixed thoroughly until a homogenous paste was achieved.

The paste was put into a mold and left to cure at room temperature under pressure applied by a hydraulic bench press at 6-8 tons of pressure for 2 days. The resulting plugs were released from the mold.

Tacrolimus constituted 9% of the finished plug by dry weight.

### Example 6: In Vitro Release of Tacrolimus from Composite Punctal Plugs

2 plugs (0.75 mg each) were placed in an Eppendorf tube containing 0.5 mL of a release medium (0.005% benzalkonium chloride, 0.1% Triton X-100 in PBS). The tubes were incubated at 37° C. on an orbital shaker at 50 rpm.

At designated time points occurring after 1, 3, 5, 9, 20, 30, 47, 60, and 93 days, the plugs were transferred from the current tube to a new tube containing the same quantity of fresh release medium and incubation continued under the same conditions.

The supernatants from each sample were injected into an HPLC machine. The HPLC machine was configured to operate as follows:
HPLC running conditions:
Instrument: Waters 2695 separation module coupled to Waters 996 PDA detector
Column: Kinetex 100 Å, C18 4.6×250 mm 5 μm
Mobile Phase A: ACN:Water:H₃PO₄=700:300:0.2
Diluent:ACN: Water50:50
Column temperature: 60° C. +/−5° C.
Sample Temperature: Ambient
Injection volume: 20 μL
Detector: PDA at 200 nm
Standard calibration curve showed linearity between 1 μg/ml to 20 mg/ml

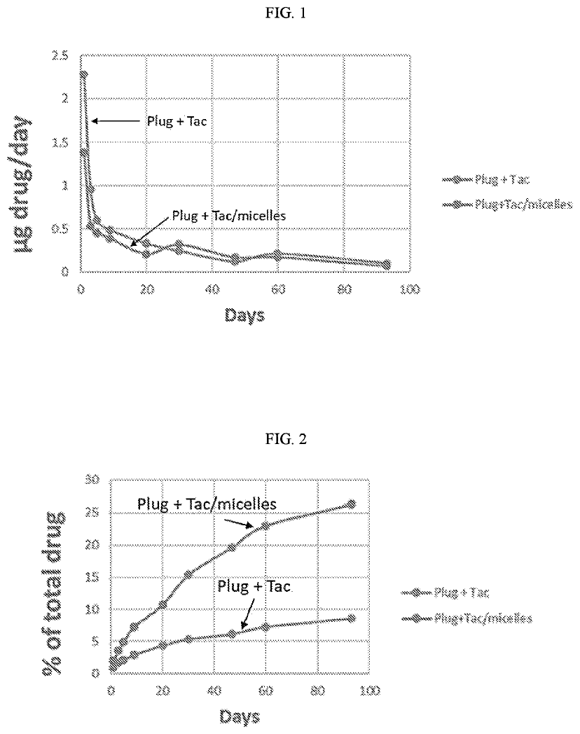

FIG. 1 shows the mass of tacrolimus release per day, over a 60-day period, for punctal plugs containing free tacrolimus prepared in accordance with Example 4 (shown in blue) and for punctal plugs containing tacrolimus-loaded micelles prepared in accordance with Example (shown in red).

FIG. 2 shows the accumulation of percentage of total mass of tacrolimus released, over a 60-day period, for punctal plugs containing free tacrolimus prepared in accordance with Example 4 (shown in blue) and for punctal plugs containing tacrolimus-loaded micelles prepared in accordance with Example 5 (shown in red). It may be seen that the percentage of total mass of tacrolimus released by punctal plugs containing tacrolimus-loaded micelles after 60 days exceeded 25%, while the percentage of total mass of tacrolimus released by punctal plugs containing free tacrolimus after 60 days was less than 10%.

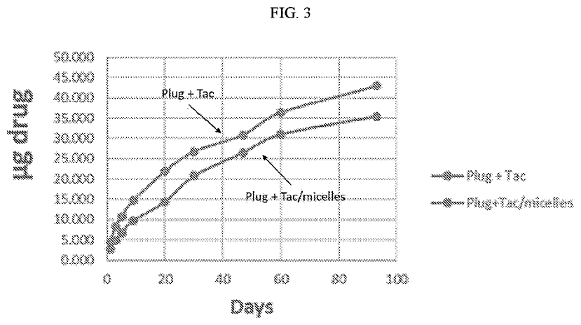

FIG. 3 shows the accumulation of total mass of tacrolimus released, over a 60-day period, for punctal plugs containing free tacrolimus prepared in accordance with Example 4 (shown in blue) and for punctal plugs containing tacrolimus-loaded micelles prepared in accordance with Example 5 (shown in red). It may be seen that the total mass of tacrolimus released by punctal plugs containing tacrolimus-loaded micelles after 60 days exceeded 35 micrograms, while the percentage of total mass of tacrolimus released by punctal plugs containing free tacrolimus after 60 days exceeded 40 micrograms.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

### Exemplary Embodiments

The above disclosure may be understood with reference to the following non-limiting exemplary embodiments.

Embodiment 1: A device, comprising:
a quantity of micelles containing an active pharmaceutical ingredient;
a quantity of fumed silica; and
a quantity of a binder,
wherein the device is in the form of a punctal plug, and
wherein the device includes between 5% and 30% of the active pharmaceutical ingredient by weight.

Embodiment 2: The device of embodiment 1, further comprising a quantity of a clay.

Embodiment 3: The device of embodiment 2, wherein the clay is kaolin.

Embodiment 4: The device of embodiment 2, wherein the device includes between 5% and 15% of the clay by weight.

Embodiment 5: The device of embodiment 1, wherein the device includes between 10% and 30% of the fumed silica by weight.

Embodiment 6: The device of embodiment 1, wherein the binder is an epoxy.

Embodiment 7: The device of embodiment 1, wherein the device includes between 20% and 60% of the binder by weight.

Embodiment 8: A method, comprising:
providing micelles containing an active pharmaceutical ingredient;
suspending fumed silica in an alcohol to produce a suspension;
drying the suspension to produce dried silica;
milling the dried silica to produce milled silica;
mixing the micelles, the milled silica, and an epoxy resin to produce a paste;
placing the paste in a mold; and
curing the molded paste to produce punctal plugs.

Embodiment 9: The method of embodiment 8, wherein the step of providing the micelles containing the active pharmaceutical ingredient comprises sub-steps of:
preparing a dispersion of a surfactant in a buffer;
dissolving the active pharmaceutical ingredient in an alcohol to produce an active pharmaceutical ingredient solution;
mixing the active pharmaceutical ingredient solution with the dispersion and adding an additional quantity of an alcohol to produce a mixture;
without alcohol; and
stirring the mixture until the alcohol evaporates to produce the mixture freezing the mixture without alcohol and lyophilizing to produce the micelles containing the active pharmaceutical ingredient.

1. A device, comprising:
a plurality of micelles containing an active pharmaceutical ingredient;
a quantity of fumed silica; and
a quantity of a binder,
wherein the device is in the form of a punctal plug, and
wherein the device includes between 5% and 30% of the active pharmaceutical ingredient by weight.

2. The device of embodiment 1, further comprising a quantity of a clay.

3. The device of embodiment 2, wherein the clay is kaolin.

4. The device of embodiment 2, wherein the device includes between 5% and 15% of the clay by weight.

5. The device of embodiment 1, wherein the device includes between 10% and 30% of the fumed silica by weight.

6. The device of embodiment 1, wherein the binder is an epoxy.

7. The device of embodiment 1, wherein the device includes between 20% and 60% of the binder by weight.

8. A method, comprising:
providing a plurality of micelles containing an active pharmaceutical ingredient;
suspending fumed silica in an alcohol to produce a suspension;
drying the suspension to produce dried silica;
milling the dried silica to produce milled silica;
mixing the micelles, the milled silica, and an epoxy resin to produce a paste;
placing the paste in a mold; and
curing the molded paste to produce punctal plugs.

9. The method of embodiment 8, wherein the step of providing the micelles containing the active pharmaceutical ingredient comprises sub-steps of:
preparing a dispersion of a surfactant in a buffer;
dissolving the active pharmaceutical ingredient in an alcohol to produce an active pharmaceutical ingredient solution;
mixing the active pharmaceutical ingredient solution with the dispersion and adding an additional quantity of an alcohol to produce a mixture;
stirring the mixture until the alcohol evaporates to produce the mixture without alcohol; and
freezing the mixture without alcohol and lyophilizing to produce the micelles containing the active pharmaceutical ingredient.
